(19) United States
(12) Patent Application Publication
(10) Pub. No.: US 2023/0149540 A1
(43) Pub. Date: May 18, 2023

(54) VACCINE PLATFORM FOR THE INDUCTION OF SYSTEMIC IMMUNE RESPONSES

(71) Applicant: The Board of Trustees of the Leland Stanford Junior University, Stanford, CA (US)

(72) Inventors: Thomas L. Cherpes, Stanford, CA (US); Rodolfo D. Vicetti Miguel, Stanford, CA (US)

(21) Appl. No.: 17/916,942

(22) PCT Filed: Apr. 6, 2021
(86) PCT No.: PCT/US2021/026022

§ 371 Date: Oct. 4, 2022

Related U.S. Application Data
(60) Provisional application No. 63/005,734, filed on Apr. 6, 2020.

(51) Int. Cl. A61K 39/39; A61K 39/00; A61K 39/395; A61K 38/17; C07K 16/28; A61P 35/00

(52) U.S. Cl. CPC A61K 39/39; A61K 39/00119; A61K 39/3955; A61K 38/1774; C07K 16/2803; A61P 35/00; A61K 2039/55555; A61K 2039/55516

(57) ABSTRACT

Compositions and methods are provided relating to vaccine formulations comprising (i) an agent that specifically binds to CD244; (ii) an effective dose of an antigen; and (iii) an adjuvant, which adjuvant can be, without limitation, an activator of innate-like T cells.

### CROSS-REFERENCE TO RELATED APPLICATIONS

This application is a 371 Application and claims the benefit of PCT Application No. PCT/US2021/026022, filed Apr. 6, 2021, which claims the benefit of U.S. Provisional Patent Application No. 63/005,734, filed Apr. 6, 2020, which applications are incorporated herein by reference in their entireties.

### BACKGROUND

Long-term immune responses are mediated by antigen-specific lymphocytes and antibodies that are formed upon exposure to pathogens or vaccines. During an initial response, interactions of specific classes of immune cells (e.g. lymphocytes and dendritic and other antigen-presenting cells), memory B and T cells are generated, which able to induce a rapid and powerful recall response. Although most vaccines have focused on humoral immunity—the generation of neutralizing antibodies, these vaccines can be ineffective against chronic infections and treatment of cancer. Rather, studies have indicated that induction of strong T cell immune responses can be required for prevention and treatment of these conditions.

The development of T cell-directed vaccines has gained increasing attention. However, the critical factors to develop T cell-mediated immune responses have not been clearly defined. Thus, more knowledge is required to tailor a vaccine's capacity to induce durable CD4⁺ and/or CD8⁺ T cell responses of appropriate magnitude and quality to effectively contribute to pathogen or tumor cell clearance. Elucidating the mechanisms through which antigen-specific T cell populations mediate long-term protection remains an important goal and can facilitate the development of effective and safe T cell-directed vaccines.

### INCORPORATION BY REFERENCE OF SEQUENCE LISTING

A Sequence Listing is provided herewith as a Sequence Listing text, STAN-1708_SEQLIST_ST25, created on Oct. 3, 2022 and having a size of 37,182 bytes. The contents of the Sequence Listing text are incorporated herein by reference in their entirety.

### SUMMARY OF THE INVENTION

Vaccine compositions are provided that, when administered to a host mammal, promote immune responses to a targeted antigen, e.g. to promote an enhanced T cell response. The vaccine compositions comprise (i) an agent that specifically binds to CD244; (ii) an effective dose of an antigen; and (iii) an adjuvant, which adjuvant can be, without limitation, an activator of innate-like T cells. In some embodiments the composition is a particle comprising each of components (i), (ii), and (iii). The particles may be provided in a pharmaceutically acceptable excipient.

Enhanced T cell responses can be one or both of antigen-specific CD4⁺ T cell responses and antigen-specific CD8⁺ T cell responses. Proliferation and activation of innate-like T cells can also result from administration of the vaccine. Enhancement of B cell responses and antibody production specific for the antigen can also result from administration of the vaccine.

In some embodiments, the agent that specifically binds to CD244 is an antibody. In some embodiments the antibody specifically binds to human CD244. The antibody may be present as an intact antibody, i.e. comprising variable and constant region sequences, or may be provided as a variable region polypeptide, e.g. scFv, F(Ab), F(Ab′), F(Ab′)s, etc. fragments. The antibody may be humanized, human, chimeric with human constant region sequences, etc. Optionally the antibody is a CD244 agonist antibody.

In other embodiments the agent that specifically binds to CD244 is a CD48 polypeptide or binding fragment thereof. The CD48 polypeptide may be a human CD48 polypeptide or a CD48 polypeptide from a species that cross-reacts with human CD244. The polypeptide may comprise the binding domain of human CD48 fused to a Fc region of human IgG. The polypeptide may consist of the binding domain of human CD48 fused to His tag. The polypeptide may consist of the binding domain of human CD48 fused to a flexible linker, e.g. SEQ ID NO:4, SEQ ID NO:5.

The antigen component may be a polypeptide, carbohydrate, lipid, etc. antigen. The antigen is present at an effective dose on the particle sufficient to provide for an antigen-specific response. In some embodiments the antigen is a protein, including without limitation a tumor-associated protein, a bacterial pathogen protein, a viral pathogen protein, a protozoan pathogen protein, etc. Antigenic polypeptides can range in size from full-length proteins to polypeptides greater than about 8 amino acids.

In some embodiments the activator of innate-like T cells is an MHC-related protein and antigen recognized by the targeted population of innate-like T cells. In some embodiments the targeted population of innate-like T cells are mucosal-associated invariant T (MAIT) cells; and the MHC-related protein is MR1. In some such embodiments the MR1 protein is human for targeting to human MAIT cells. In some embodiments the antigen is a microbial-derived metabolite. In other embodiments the targeted population of innate-like T cells are invariant natural killer T (iNKT) cells, and the MHC-related protein is CD1d. In some such embodiments the CD1d protein is human for targeting to human iNKT cells. In some such embodiments the antigen is α-galactosylceramide (α-GalCer). In some embodiments the activators of innate-like T cells is an agonist antibody that binds to the TCR of innate-like T cells. In some such embodiments the antibody targets human MAIT cells. In some such embodiments the antibody targets human NKT cells.

In some embodiments the vaccine composition is a biodegradable microparticle comprising each of components (i), (ii), and (iii). Each of (i), (ii), and (iii) may be encapsulated within a biodegradable microparticle or may be displayed on the surface of a microparticle. In some embodiments, component (i) and (iii) are displayed on the surface. In some embodiments, component (ii) is encapsulated in a biodegradable microparticle, which may be referred to herein as targeted microparticles (TMPs).

In some embodiments the vaccine composition comprises a biodegradable microparticle from about 0.05 μm in diameter to about 5 μm in diameter, and may be from about 0.1 μm to about 0.5 μm in diameter, from about 0.1 to about 0.5 μM, or from about 1 μm in diameter to about 3 μm in diameter. In certain embodiments the microparticle is comprised of poly(lactic acid) (PLA), poly(glycolic acid) (PGA), or a combination thereof (PLGA).

In some embodiments, methods are provided for stimulating an immune response, e.g. a T cell mediated response, to an antigen of interest, the method comprising administering to an individual mammal an effective dose or series of doses of a vaccine composition comprising (i) an agent that specifically binds to CD244; (ii) an effective dose of the antigen of interest; and (iii) an adjuvant, e.g. an activator of innate-like T cells. In some embodiments the composition is a particle comprising each of components (i), (ii), and (iii). The particles may be provided in a pharmaceutically acceptable excipient.

Other aspects and features will be readily apparent to the ordinarily skilled artisan upon reading the present disclosure.

### BRIEF DESCRIPTION OF THE DRAWINGS

The invention is best understood from the following detailed description of exemplary embodiments when read in conjunction with the accompanying drawings. It is emphasized that, according to common practice, the various features of the drawings are not necessarily to-scale. On the contrary, the dimensions of the various features are arbitrarily expanded or reduced for clarity. Included in the drawings are the following figures:

FIG. 1. Identification of specific receptors targeted by human Chlamydia-activated B cells (CABs) using cell microarray technology. Screening studies used a library of expression vectors containing open reading frames encoding full-length human plasma membrane proteins. Using this approach, 4 receptors that interact specifically with human CABs were discovered. Two had strong binding (CD244 and CTLA4), one (CD70) had moderate binding, and one (ICOSLG) had weak binding.

FIG. 2. CAB binding to SIRPα is demonstrated to be non-specific, as an unrelated human cell line (HEK293T) also was able to bind this receptor.

FIG. 3. Murine type 1 conventional dendritic cell (cDC) expression of receptors identified by screening assay described in FIG. 1. Evaluation of the expression of the identified receptors in mouse DCs demonstrated that splenic cDCs (type 1 and type 2) from C57BL/6 mice displayed high levels of CD244, intermediate levels of ICOSLG, and negligible levels of CTLA4 and CD70. Mouse splenic CD8⁺ DCs were identified as live CD90⁻ B220⁻ MHC-II⁺ CD11c⁺ CD8⁺ cells.

FIG. 4. Expression of CD244 by human type 2 cDCs. The data show that human cDC express high levels of surface CD244. Human CD1c⁺ DCs from PBMCs were identified as live CD3ε⁻ CD14⁻ CD16⁻ CD20⁻ HLA-DR⁺ CD11c⁺ CD1c⁺ cells.

FIG. 5. Expression of CD48, the ligand for CD244, on mouse Chlamydia-activated B cells (CABs). Mouse Chlamydia-activated B cells were identified as live CD90− B220+ MHC-II+ cells.

FIG. 6. Schematic of interactions between targeted microparticles, dendritic cells, innate-like T cells and CD8⁺ T cells. Targeted microparticles (TMPs) are designed to be 0.1-3 μm diameter, comprising surfaces decorated with an antibody or ligand that bind surface receptors for DC targeting; contain sufficient antigen to induce the desired response, and have an adjuvant, which in this case has the capacity to deliver a signal that activates the innate-like T cells of interest.

FIG. 7. In vitro exposure of human or rhesus macaque MAIT cells to their cognate antigen (5-OP-RU) (a highly unstable molecule) or microspheres loaded with 5-OP-RU-loaded MR-1 monomer induced IFN-γ secretion. Representative pseudocolor plots shown are gated on live CD8⁺ T cells. Human and rhesus macaque cells were stimulated for 18 h.

FIG. 8. Using TMPs to activate NKT cells in vivo. A. Design of animal study in which mice were 1) left untreated, or were intravenously injected with 2) CABs loaded with αGC to induce iNKT cell expansion (positive control) or 3) CD1d-αGC-coated microspheres (test group). B. Three days later, they splenocytes were obtained to determine the frequency of NKT cells using flow cytometry. Contour plots gated on live CD90⁺ splenocytes. C. CD1d-αGC-coated microspheres were capable of inducing a sizable expansion of NKT cells not only in the spleen, but also in lungs and liver. Mice administered a single dose of microspheres loaded with an innate-like T cell ligand displayed robust in vivo expansion of iNKT cells in the spleen, lungs and liver.

FIG. 9. Single TMP vaccination primes antigen-specific CD8+ T cells and expands NKT cells. A. Design of study in which fluorescently (CTV)-labeled ovalbumin-specific CD8⁺ T cells (Vα2⁺ cells) were transferred from TCR-transgenic mice into wild type C57BL/6 mice prior to administration of 1) antigen-loaded CABs that were or were not loaded with αGC; 2) TMPs covered with an anti-CD244 monoclonal antibody (clone (B6)458.1) and loaded only with the antigen; or 3) TMPs covered with the same anti-CD244 monoclonal antibody and loaded with the antigen and αGC-loaded CD1d monomers. B. anti-CD244-decorated TMPs primed robust antigen-specific CD8⁺ T cells responses and induced activation of NKT cells for optimal effector function of primed CD8⁺ T cells. Contour plots gated on live CD8⁺ T cells. C. Single TMP injection induced splenic NK T cell expansion. Contour plots gated on live CD90⁺ splenocytes.

FIG. 10. Single TMP vaccination primes antigen-specific CD4⁺ and CD8⁺ T cells. A. Design of study in which fluorescently labeled ovalbumin-specific CD4⁺ and CD8⁺ T cells were transferred from TCR-transgenic mice to wild type mice 1 day prior to vaccination with antigen-loaded CABs or TMPs displaying DC-targeting monoclonal antibodies or a recombinant CD48-Fc chimeric protein (controls mice received no vaccination). Three days after vaccination, mice were euthanized and spleens obtained to evaluate proliferation of antigen-specific CD4⁺ and CD8⁺ T cells. B. CABs did not prime CD4⁺ T cells in vivo; TMPs loaded with anti-CD244 mAb (clone (B6)458.1) and antigen induced robust proliferation of antigen-specific CD4⁺ T cells. C. Mice were vaccinated with antigen-loaded CABs, antigen-loaded TMPs decorated with either anti-CD205 mAb (clone NLDC-145), anti-CD244 mAb or recombinant mouse CD48 fused to human IgG1 (CD48-Fc). Anti-CD205 mAb-loaded TMPs and anti-CD244 mAb-loaded TMPs generated similar levels of activated antigen-specific CD8⁺ T cells, while CD48 for targeting of TMPs to DCs demonstrated greater capacity to promote antigen-specific CD8⁺ T cells in vivo.

FIG. 11. CD48-loaded TMP can induce therapeutic tumor-specific CD8⁺ T cell responses. A. TMPs loaded with recombinant mouse CD48 fused to human IgG1, immunodominant peptide of HPV16 E7 and αGC-loaded CD1d monomers to induce robust antigen-specific CD8⁺ T cell responses. Contour plots gated on live CD8⁺ T cells. TMP-based vaccination 7d prior. B. A single intravenous administration of TMPs loaded with recombinant mouse CD48 fused to human IgG1, immunodominant peptide of HPV16 E6 and E7 and αGC-loaded CD1d monomers induced complete rejection of established TC-1 tumors, which cells express HPV16 E6 and E7.

FIG. 12. CD48-loaded TMP can prevent the establishment of pulmonary tumor foci. Mice were vaccinated with a single dose of TMPs loaded with a recombinant mouse CD48 fused to human IgG1, immunodominant peptides for TRP2 and gp100 (melanoma-associated antigens) and αGC-loaded CD1d monomers or were left untreated. Thirty days later, both groups of mice were intravenously injected with 2×10⁵ B16.F10 melanoma cells. A. In one experiment, mice were euthanized 18 days after tumor injection, and the number of pulmonary tumor foci was determined. B. In another experiment, mice were followed after tumor injection to determine overall survival in both groups. Single TMP vaccination induced tumor-specific memory CD8⁺ T cells responses capable of preventing pulmonary tumor establishment.

FIG. 13. Human CD48-decorated TMP can prime antigen-specific CD8⁺ T cell responses in vivo. TMPs loaded with recombinant mouse CD48 with a His tag or loaded with recombinant human CD48 fused to human IgG1, were also concomitantly loaded with the immunodominant peptide of HPV16 E7 and αGC-loaded CD1d monomers to induce robust antigen-specific CD8⁺ T cell responses. Contour plots gated on live CD8⁺ T cells. TMP-based vaccination 7d prior.

FIG. 14. Sequences of relevant CD48 proteins are provided as SEQ ID NO:1 (human), (SEQ ID NO:2) Macaque and (SEQ ID NO:3) mouse.

### DETAILED DESCRIPTION OF THE EMBODIMENTS

It is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

By the term “vaccine” as used herein, is meant a composition comprising (i) an agent that specifically binds to CD244; (ii) an effective dose of an antigen; and (iii) an adjuvant, e.g. an activator of innate-like T cells, which, when administered to a subject, induces cellular or humoral immune responses as described herein.

Some embodiments of the invention provide a method of stimulating an immune response in a mammal, which can be a human or a preclinical model for human disease, e.g. mouse, ape, monkey etc. “Stimulating an immune response” includes, but is not limited to, inducing a therapeutic or prophylactic effect that is mediated by the immune system of the mammal. More specifically, stimulating an immune response in the context of the invention refers to eliciting cellular or humoral immune responses, thereby inducing downstream effects such as production of antibodies, antibody heavy chain class switching, maturation of APCs, and stimulation of cytolytic T cells, T helper cells and both T and B memory cells.

As appreciated by skilled artisans, vaccine compositions are suitably formulated to be compatible with the intended route of administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of the composition can be adjusted with acids or bases, such as hydrochloric acid or sodium hydroxide. Systemic administration of the composition is also suitably accomplished by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

Vaccine compositions may include an aqueous medium, pharmaceutically acceptable inert excipient such as lactose, starch, calcium carbonate, and sodium citrate. Vaccine compositions may also include an adjuvant, for example Freud's adjuvant. Vaccines may be administered alone or in combination with a physiologically acceptable vehicle that is suitable for administration to humans. Vaccines may be delivered orally, parenterally, intramuscularly, intranasally or intravenously. Oral delivery may encompass, for example, adding the compositions to the feed or drink of the mammals. Factors bearing on the vaccine dosage include, for example, the weight and age of the mammal. Compositions for parenteral or intravenous delivery may also include emulsifying or suspending agents or diluents to control the delivery and dose amount of the vaccine.

The term “immune response” refers to any response to an antigenic or immunogenic compound by the immune system of a vertebrate subject. Exemplary immune responses include but are not limited to local and systemic cellular as well as humoral immunity, such as cytotoxic T lymphocytes (CTL) responses, including antigen-specific induction of CD8⁺ CTLs, CD4⁺ helper T-cell responses including T-cell proliferative responses and cytokine release, and B-cell responses including antibody response.

The term “eliciting an immune response” is used herein generally to encompass induction and/or potentiation of an immune response.

The term “inducing an immune response” refers to an immune response that is stimulated, initiated, or induced.

The term “potentiating an immune response” refers to a pre-existing immune response that is improved, furthered, supplemented, amplified, enhanced, increased or prolonged.

The expression “enhanced immune response” or similar means that the immune response is elevated, improved or enhanced to the benefit of the host relative to the prior immune response status, for example, before the administration of an immunogenic composition of the invention.

The terms “humoral immunity” and “humoral immune response” refer to the form of immunity in which antibody molecules are produced in response to antigenic stimulation.

The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response normally includes lymphocyte proliferation. When “lymphocyte proliferation” is measured, the ability of lymphocytes to proliferate in response to a specific antigen is measured. Lymphocyte proliferation is meant to refer to B cell, T-helper cell or cytotoxic T-lymphocyte (CTL) cell proliferation.

The term “immunogenic amount” refers to an amount of antigenic compound sufficient to stimulate an immune response, when administered with a subject immunogenic composition, as compared with the immune response elicited by the antigen in the absence of the polynucleotide adjuvant.

The term “effective dose” or “effective dosage” is defined as an amount sufficient to achieve or at least partially achieve the desired effect. The term “therapeutically effective dose” is defined as an amount sufficient to induce an immune response to the antigen and may at least at least partially arrest an infectious disease or cancer and its complications in a patient already suffering from the disease. Amounts effective for this use will depend upon the severity of the disorder being treated and the general state of the patient's own immune system.

“Polypeptide” and “protein” as used interchangeably herein, can encompass peptides and oligopeptides. Where “polypeptide” is recited herein to refer to an amino acid sequence of a naturally-occurring protein molecule, “polypeptide” and like terms are not necessarily limited to the amino acid sequence to the complete, native amino acid sequence associated with the recited protein molecule, but instead can encompass biologically active variants or fragments, including polypeptides having substantial sequence similarity or sequence identify relative to the amino acid sequences provided herein. In general, fragments or variants retain a biological activity of the parent polypeptide from which their sequence is derived. Polypeptides may be, for example, at least 8 amino acids in length, at least 10, at least 12, at least 14, at least 16, at least 18, at least 20, at least 22, at least 24, and may be at least 30, at least 40, at least 50, at least 75, at least 100 or more amino acids in length.

Polypeptides suitable for use can be obtained from any species, e.g., mammalian or non-mammalian (e.g., reptiles, amphibians, avian (e.g., chicken)), particularly mammalian, including human, rodent (e.g., murine or rat), bovine, ovine, porcine, murine, or equine, particularly rat or human, from any source whether natural, synthetic, semi-synthetic or recombinant. In general, polypeptides comprising a sequence of a human polypeptide are of particular interest.

The term “derived from” indicates molecule that is obtained directly from the indicated source (e.g., when a protein directly purified from a cell, the protein is “derived from” the cell) or information is obtained from the source, e.g. nucleotide or amino acid sequence, from which the molecule can be synthesized from materials other than the source of information.

The term “isolated” indicates that the recited material (e.g, polypeptide, nucleic acid, etc.) is substantially separated from, or enriched relative to, other materials with which it occurs in nature (e.g., in a cell). A material (e.g., polypeptide, nucleic acid, etc.) that is isolated constitutes at least about 0.1%, at least about 0.5%, at least about 1% or at least about 5% by weight of the total material of the same type (e.g., total protein, total nucleic acid) in a given sample.

The terms “subject” and “patient” are used interchangeably herein to mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest. As will be evidence from the context in which the term is used, subject and patient refer to a subject or patient susceptible to infection.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the host.

A “pharmaceutically acceptable excipient”, “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is usually free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by cell culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made in an immortalized B cell or hybridoma thereof, may be made by recombinant DNA methods, including without limitation yeast display.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

By “solid phase” is meant a non-aqueous matrix, e.g. a particle, to which the vaccine components can adhere, be conjugated to, or be encapsulated within.

An “effective amount of an antigenic compound” refers to an amount of antigenic compound which, in optional combination with an adjuvant, will cause the subject to produce a specific immunological response to the antigenic compound.

### CD244 Binding Agent

CD244 is an Ig Superfamily Signaling Lymphocyte Activation Molecule (SLAM) family receptor. Like all SLAM family receptors, it is a transmembrane receptor comprised of an extracellular segment with two immunoglobulin (Ig)-like domains, a transmembrane region, and a cytoplasmic domain containing tyrosine-based motifs. Unlike other SLAM family receptors, it does not act as a self-ligand; instead, it binds CD48, a transmembrane receptor ubiquitously expressed on hematopoietic cells. Its cytoplasmic domain includes four Immunoreceptor Tyrosine-based Switch Motifs (ITSMs) that interact with a variety of specific adaptor molecules and are capable of propagating both inhibitory and activating signals.

The reference sequence for human CD244 may be accessed at Genbank, refseq NP_057466. Antibodies to human CD244 are known in the art and commercially available from multiple sources, for example clone 2B4.69; AF1039; clone (7D24); clone C1.7; MA5-16486; eBioPP35; NBP1-76558; Clone 2-69; etc.

The natural ligand of CD244, CD48, may be accessed at Genbank NP_001769. See, for example Vaughan et al. (1991) Immunogenetics 33 (2), 113-117. Constructs of CD48 may be truncated to delete signal, IgC2 domain and GPI anchor sequences, and may comprise a flexible linker for attachment to the microparticle. Sequences of relevant CD48 proteins are provided as SEQ ID NO:1 (human), (SEQ ID NO:2) Macaque and (SEQ ID NO:3) mouse. The constructs provided herein (SEQ ID NO:4-13) provide examples of useful proteins for this purpose, including particularly SEQ ID NO:4 and SEQ ID NO:5.

Desirably an antibody specific for CD244, or CD48 protein, will be displayed on the surface of a particle to enhance binding efficacy. A polypeptide linker may be used at the terminus of the binding agent.

In some embodiments an affinity agent, e.g. biotin/avidin or streptavidin, etc. is used to link the CD244 binding agent to the microparticle. In other embodiments the microparticle is derivatized to allow for a stable linkage to the binding agent.

The CD244 binding agent may be linked through a homo- or heterobifunctional linker having a group at one end capable of forming a stable linkage to the particle surface, and a group at the opposite end capable of forming a stable linkage to the protein. Illustrative entities include: azidobenzoyl hydrazide, N-[4-(p-azidosalicylamino)butyl]-3′-[2′-pyridydithio]propionamide), bis-sulfosuccinimidyl suberate, dimethyladipimidate, disuccinimidyltartrate, N-.gamma.-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4[N-maleimidomethyl]cyclohexane-1-carboxylate; 3-(2-pyridyldithio)propionic acid N-hydroxysuccinimide ester (SPDP); N,N′-(1,3-phenylene) bismaleimide; N,N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenylsulf one (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

### Antigens

As used herein, the term “antigenic compound” refers to any substance that can be recognized by the immune system (e.g., bound by an antibody or processed so as to elicit a cellular immune response) under appropriate conditions.

An “antigen” as used herein includes but is not limited to cells; cell extracts; proteins; lipoproteins; glycoproteins; nucleoproteins; polypeptides; peptides; polysaccharides; polysaccharide conjugates; peptide mimics of polysaccharides; lipids; glycolipids; carbohydrates; viruses; viral extracts; bacteria; bacterial extracts; fungi; fungal extracts; multicellular organisms such as parasites; and allergens. In some embodiments of the invention the antigen is a polypeptide, e.g. a native polypeptide; a polypeptide produced by recombinant methods, including in vitro cell free synthesis, bacterial and prokaryotic expression systems; and the like. Such antigens include, without limitation, viral antigens derived from HIV; influenza, smallpox (vaccinia), measles, mumps, rubella, poliovirus, rotavirus, varicella (chickenpox), hepatitis A, B, C, D virus, bacterial antigens, tumor antigens, and the like. Bacterial antigens of interest include, without limitation, antigens derived from Bacillus anthracis; Bordetella pertussis, Clostridium tetani, Haemophilus Influenzae, Corynebacterium diphtheriae, Meningococcus sp., Streptococcus pneumoniae, Salmonella typhi, Mycobacterium tuberculosis, etc.

Antigens may be exogenous (e.g., from a source other than the individual to whom the antigen is administered, e.g., from a different species) or endogenous (e.g., originating from within the host, e.g., a diseased element of body, a cancer antigen, a virus infected cell producing antigen, and the like). Antigens may be native (e.g., naturally-occurring); synthetic; or recombinant. Antigens include crude extracts; whole cells; and purified antigens, where “purified” indicates that the antigen is in a form that is enriched relative to the environment in which the antigen normally occurs and/or relative to the crude extract, for example, a cultured form of the antigen. The present invention is directed to a composition further comprising an antigen or an antigenic peptide (e.g., epitope). Preferably, the antigen or antigenic peptide is recognized by autologous T cells. Any antigen may be used in the present invention that is displayed or detected on the surface of tumorous or infected cells. Such antigens include both foreign and self antigens. In many cases, a patient will recognize such antigens a “non-self” or foreign. The antigen may be a wild type antigen or mutated relative to its wild type; or may be differentially post-translationally modified relative to the wild type.

The antigen may be a self-antigen or foreign antigen. It an embodiment of the invention, the antigen is a tumor-associated antigen, such as a cancer-testes associated antigen. The antigen may be a neoantigen, and specifically a cancer neoantigen. Cancer neoantigens are tumor-specific antigens generated from gene mutations occurring in tumor cells. There are patient-specific somatic mutations occurring during neoplastic transformation and are particularly useful in the present invention.

Specific cancer antigens include for melanoma: Tyrosinase, Tyrosinase-related protein (Trp-1), gp100, Melan/MART-1; prostate adenocarcinoma; Prostate-specific membrane antigen, Prostate-specific acid phosphatase, Prostate-specific antigen; pancreatic, lung, breast and colon adenocarcinoma: MUC1; non-small-cell lung carcinoma: MUC1, MAGE antigens, EGFR; cancer/testis antigens: LAGE/NY-ESO1, MAGE antigens, CEA, AFP; breast cancer: HER-2; acute myelogenous leukemia: Aurora-A kinase, BRAP, Cyclin A1, hTert, WT1, chronic lymphocytic leukemia: ROR1; chronic myelogenous leukemia: BCR/ABL, BRAP, CML28, CML66, PR1, Proteinase 3, survivin, WT1.

Antigens recognized by T cells, whether helper T lymphocytes or CTL, are not recognized as intact proteins, but rather as small peptides that associate with class I or class II MHC proteins on the surface of cells. During the course of a naturally occurring immune response, antigens that are recognized in association with class I or II MHC molecules on antigen presenting cells (APCs) are acquired from outside the cell, internalized, and processed into small peptides that associate with the class I or II MHC molecules.

Antigens that give rise to proteins that are recognized in association with class I MHC molecules are generally proteins that are produced within the cells, and these antigens are processed and associate with class I MHC molecules. It is now understood that the peptides that associate with given class I or class II MHC molecules are characterized as having a common binding motif, and the binding motifs for a large number of different class I and II MHC molecules have been determined. Synthetic peptides can also be synthesized that correspond to the amino acid sequence of a given antigen and that contain a binding motif for a given class I or II MHC molecule. These peptides can then be added to appropriate APCs, and the APCs can be used to stimulate a T helper cell or CTL response either in vitro or in vivo. The binding motifs, methods for synthesizing the peptides, and methods for stimulating a T helper cell or CTL response are all known and readily available to one of ordinary skill in the art.

In an embodiment of the invention, the antigen is a peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-Catenin, CDK4, Mum-1, p16, TAGE, PSMA, PSCA, CT7, telomerase, 43-9F, 5T4, 791Tgp72, alpha-fetoprotein, beta-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, b-amyloid, CA125, CD40, EGFR, G17DT, GD2/3L, gp100, IMA950, KOC1, Peptidyl arginine deiminase-4, MUC-1, OFA, PANVAC, PAP, PSA, PSMA, SL701, SSX-2, TTK, TACAS, URLC10, vEGFR, WT-1. In one embodiment, the antigen is selected from patient specific neoantigens or β-amyloid protein or tumor antigen with high mutation loads.

In another embodiment, the antigen is present on the cancer cells of a patient suffering from cancer, such as melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer.

Cancer antigen vaccines may be administered in combination with other agents and antibodies known for cancer treatment, including, for example, checkpoint inhibitor antibodies.

Compositions comprising an antigen protein or peptide are, or can be, made synthetically or by purification from a biological source. They can be made recombinantly. Desirably they are in some embodiments at least 90% pure, in some embodiments at least 92% pure, in some embodiments at least 93% pure, in some embodiments at least 94% pure, in some embodiments at least 95% pure, in some embodiments at least 96% pure, in some embodiments at least 97% pure, in some embodiments at least 98% pure, and in some embodiments at least 99% pure. For administration to a human, they generally do not contain other components that might be harmful to a human recipient.

Under certain circumstances it can be desirable to add additional antigenic proteins or antigenic peptides to the composition, for example, to make a cocktail having the ability to stimulate an immune response in a number of different HLA type hosts. Alternatively, additional proteins and/or peptides can provide an interacting function within a single host, such as but not limited to an adjuvant function or a stabilizing function. As a non-limiting example, tumor antigens can be used in admixture with the antigen peptides such that multiple different immune responses are induced in a single patient.

### Adjuvants and Activators of Innate-Like T Cells

The term “adjuvant” or “vaccine adjuvant” as used herein refers to any substance or combination of substances which non-specifically enhances the immune response to an antigen. Alum, ASO4, MF59, AS03, AS01 and CpG ODN are currently approved for use in human vaccines. Adjuvants as a delivery system in subunit vaccines, such as liposomes, immune stimulating complexes (ISCOMs) and nanoparticles, are considered effective in stimulating protective immunity. Such adjuvants prevent rapid degradation of proteins and peptides in vivo, thereby enhancing the dose effectiveness of the vaccine antigen.

Certain adjuvants activate TLRs including TLR2, 7, 8 and 9. Poly(I:C) and its two derivatives, polyl:C12U (Ampligen) and poly(IC:LC) (Hiltonol) have been used in clinical trials against both tumors and infectious diseases. TLR4 is targeted by monophosphoryl lipid (MPL)A. AS04 (containing MPL) is approved for use. AS01 (containing MPL) is also used in a vaccine. TLR7 and TLR8 recognizing single-stranded RNA (ssRNA) molecules are targeted by small-molecule immune potentiator (SMIP)-based adjuvants such as imiquimod and resiquimod. Intracellular NLRs such as NOD1 and NOD2 receptors recognize diaminopimelatic acid (DAP)-containing muropeptide, while NOD2 detects the muramyl dipeptide (MDP) component present in all bacterial peptidoglycans.

Adjuvants that are inducers of damage-associated molecular patterns (DAMPs) trigger innate immune responses in vivo by damaging the host cells, thereby resulting in the release of DAMP factors (ex. RNA, DNA) for subsequent activation of the innate immune receptors. The cytosolic receptor NLRP3 is recognized by adjuvants such as Quil-A and chitosan, as well as ATP, MDP, uric acid crystals and silica. These compounds generate DAMP signals, such as reactive oxygen species (ROS) or induce potassium efflux to activate NLRP3.

Carbohydrate-based adjuvants include glucans, fructans, mannans, chitin/chitosan and other carbohydrate compounds derived from Mycobacterium spp. (including lipoarabinomannan, muramyldipeptide/MDP, trehalose-6-6-dimycolate/TDM), as well as LPS and saponin compounds (including QS-21, a saponin in an oil-in-water emulsion).

In some embodiments the adjuvant is an activator of innate-like T cells. In some embodiments the activator of innate-like T cells is an MHC-related protein and antigen recognized by the targeted population of innate-like T cells. In some embodiments the targeted population of innate-like T cells are mucosal-associated invariant T (MAIT) cells; and the MHC-related protein is MR1. In some such embodiments the MR1 protein is human for targeting to human MAIT cells. In some embodiments the antigen is a microbial-derived metabolite. In other embodiments the targeted population of innate-like T cells are invariant natural killer T (iNKT) cells, and the MHC-related protein is CD1d. In some such embodiments the CD1d protein is human for targeting to human iNKT cells. In some such embodiments the antigen is α-galactosylceramide (α-GalCer). In some embodiments the activators of innate-like T cells is an agonist antibody that binds to the TCR of innate-like T cells. In some such embodiments the antibody targets human MAIT cells. In some such embodiments the antibody targets human NKT cells.

Innate-like T cells are innate lymphoid cells that have features similar to T cells involved in acquired immunity, such as T cell receptor (TCR) expression. However, their TCR repertoire is very limited, and they recognize self or exogenous non-peptide antigens presented by a family of non-polymorphic and MHC class I-related molecules. The two major classes are iNKT cells and MAIT cells.

Mucosal-associated invariant T (MAIT) cells are unconventional T cells with innate-like antimicrobial activity. MAIT cells are highly abundant in humans, representing approximately 3-5% of human blood T cells, and even higher frequency in other tissues, such as liver where they are up to 40% of T cells. MAIT cells are typically defined by their expression of an invariant T cell receptor (TCR)-α chain. In humans, this consists of TRAV1-2 joined to TRAJ33, TRAJ12 or TRAJ20 with little to no n nucleotide additions at the TCR-α complementarity determining region 3 (CDR3α) junction. This pairs with a TCR-β repertoire highly biased toward TRBV6 family members and TRBV20-1. This unique TCR has been highly conserved throughout mammalian evolution. Upon antigenic stimulation MAIT cells can undergo marked expansion to represent up to ≥50% of T cells.

The highly conserved MAIT TCR restricts MAIT cells to the recognition of the major histocompatibility class (MHC) class I-related protein MR1. Unlike classical MHC I molecules, the Ag-binding cleft of MR1 includes a small Ag-binding pocket (the A′ pocket) lined with aromatic amino acid side chains, imbuing an ability to capture and present small metabolite compounds. Several MR1-bound Ags have been described, including a range of microbial-derived vitamin B2 (riboflavin) derivatives that are antigenic for MAIT cells, such as the ribityl-lumazines 7-hydoxy-6-methyl-8-D-ribityllumazine (RL-6-Me-7-OH) and 6,7-dimethyl-8-D-ribityllumazine (RL-6,7-diMe), as well as the highly potent pyrimidine Ags such as 5-OP-RU. More recently, acetylated RL-6-Me-7-OH, the photolumazines 6-(2-carboxyethyl)-7-hydroxy-8-ribityllumazine (photolumazine I; PLI), 6-(1H-indol-3-yl)-7-hydroxy-8-ribityllumazine (photolumazine III; PLIII), the riboflavin analogue 7,8-didemethyl-8-hydroxy-5-deazariboflavin (FO) and riboflavin itself have been described as MR1-binding ligands, although riboflavin and FO were inhibitors rather than activators of MAIT cells. MR1 can also capture vitamin B9 (folate)-derivative, pterin-based molecules including 6-formyl pterin (6-FP) and its synthetic analogue Acetyl (Ac)-6-FP.

In some embodiments, the activating agent is human MR1 monomer complexed with an antigen recognized by MAIT cells. In some embodiments the activating agent is 5-OP-RU-loaded MR-1 monomer. For example, see U.S. Pat. No. 10,011,602, herein specifically incorporated by reference, which describes the monomer. In some embodiments the activator of innate-like T cells is an agonist antibody that binds to the TCR of human MAIT cells.

NKT cells are characterized by the expression of TCRs with a limited repertoire, consisting of Vα24 and Jα18 (in humans). In addition, their sets of Vβs are also skewed toward mainly V{11 (in humans). Since NKT cells have limited TCRs, they are also called invariant natural killer T (iNKT) cells. iNKT cells recognize CD1d protein complexed with antigen, for example α-galactosylceramide (α-GalCer) presented by CD1d is a ligand that activates iNKT cells. In some embodiments the activated agent is a complex of α-GalCer complexed with CD1d. For example, see US Patent publication 2017/0029454 which describes the CD1d protein and US20170312356A1, which describes ligands including PBS-57, each of which are herein specifically incorporated by reference. In some embodiments the activators of innate-like T cells is an agonist antibody that binds to the TCR of human NKT cells.

Microparticles, for example particles of 100 nm to 150 μm in diameter, from about 200 nm to about 30 μm in diameter, from about 500 nm to 10 μm in diameter, from about 500 nm to about 3 μm in diameter, are formed from materials that are biodegradable and non-toxic. The particles are optionally treated to stably join the CD244 binding agent to the surface. The antigen and the activating agent may be dispersed or encapsulated within the microparticle.

Biodegradable polymers are typically degraded into individual monomers, which are metabolized and removed from the body via normal metabolic pathways. Some preferred biodegradable polymers include poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), and poly(methacrylic acid). Biodegradable polymers particularly preferred in the present invention include polylactides (PLA), polyglycolides (PGA), poly(lactide-co-glycolides) (PLGA), polyanhydrides, polycaprolactone, poly-3-hydroxybutyrate and polyorthoesters. Such biodegradable polymers have been characterized extensively and can be formulated to exhibit desired degradation properties as (see, e.g., Edlund & Albertsson, Degradable Aliphatic Polyesters, pp. 67-112 (2002), Barman et al., J. of Controlled Release 69:337-344 (2000); Cohen et al., Pharmaceutical Res. (8): 713-720 (1991)). Degradation and drug release kinetics can be precisely controlled by the physicochemical properties of the polymer, such as molecular weight, dispersity index, hydrophobicity, and crystallinity. In general, therapeutics can be released in a controlled manner with first-order kinetics due to drug diffusion through the polymeric matrix or triggered in response to the local environment. The nanoparticle surface may be sterically stabilized by grafting, conjugating, or adsorbing hydrophilic polymers such as PEG to its surface, which can also reduce hepatic uptake and improve circulation half-life.

In one particular embodiment, the polymer comprises poly(lactide-co-glycolides) (PLGA). PLGA is a copolymer which has been used in a host of FDA approved therapeutic devices, owing to its biodegradability and biocompatibility. During polymerization, successive monomeric units of glycolic or lactic acid are linked together in PLGA by ester linkages, thus yielding a linear, aliphatic polyester as a product.

Depending on the ratio of lactide to glycolide used for the polymerization, different forms of PLGA can be obtained: these are usually identified in regard to the monomers' ratio used (e.g., PLGA 75:25 identifies a copolymer whose composition is 75% lactic acid and 25% glycolic acid). PLGA degrades by hydrolysis of its ester linkages in the presence of water. It has been shown that the time required for degradation of PLGA is related to the monomers' ratio used in production: the higher the content of glycolide units, the lower the time required for degradation. An exception to this rule is the copolymer with 50:50 monomer ratio which exhibits a faster degradation (about two months). In addition, polymers that are end-capped with esters (as opposed to the free carboxylic acid) demonstrate longer degradation half-lives. The vaccine may be encapsulated in batches of microparticles having different release profile. In such embodiments, a single type of biodegradable polymer may be used, but used in formulations with different release profiles; alternatively, different biodegradable polymers having different release characteristics may be used.

In other embodiments the particles are liposome microparticles. Lipids form microparticle vesicles through the self-assembly of amphiphilic lipids and excipients. The lipids form a bilayer based on hydrophobic interactions in continuous parallel packing, with the hydrophilic head groups positioned towards the aqueous environment. Hydrophilic molecules can be encapsulated in the inner aqueous phase while hydrophobic molecules can be carried in the hydrophobic domains of the lipid bilayer. Physicochemical properties of liposomes can be precisely changed to control surface charge, functionality, and size by simply mixing commercially available lipid molecules. Generally, lipids used to prepare vesicular formulations are found in the human body and approved by the FDA, such as DSPE (1,2-distearoyl-sn-glycero-3-phosphoethanolamine), HSPC (hydrogenated phosphatidylcholine from soybean lecithin), EggPG (egg yolk phosphatidylglycerol) and DSPC (1,2-distearoyl-glycero-3-phosphocholine). Each of these lipids can be obtained with or without polyethylene glycol (PEG), which can be used to modify the surface of the resulting liposome.

Methods of Use

In the methods disclosed herein, an immunologically effective amount of a vaccine composition as described herein is administered to a patient by administrations of a vaccine, in a manner effective to result in an improvement in the patient's condition. The timing of doses depends upon factors well known in the art. After the initial administration one or more booster doses may subsequently be administered to maintain antibody titers and efficacy of cell-mediated immunity. An example of a dosing regimen would be a dose on day 1, a second dose at from 1 to 2 months, a third dose at either 4, 6 or 12 months, and additional booster doses at distant times as needed. In one aspect, the invention provides a means for classifying the immune response to vaccine, e.g., 9 to 15 weeks after administration of the vaccine; by measuring the level of antibodies or responsive T cells against the antigen of the vaccine.

The vaccine formulations may be used in immunization for the various diseases. In some embodiments, the recipient is infected or at high risk of microbial infection. In some embodiments the recipient is suffering from cancer.

The vaccine formulation is administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the vaccine formulation is suitably administered by pulse infusion, particularly with declining doses of the vaccine.

For the prevention or treatment of disease, the appropriate dosage of vaccine will depend on the type of disease to be treated, the severity and course of the disease, whether the vaccine is administered for preventive purposes, previous therapy, the patient's clinical history and response to the vaccine, and the discretion of the attending physician. The vaccine is suitably administered to the patient at one time or over a series of treatments.

In another embodiment of the invention, an article of manufacture containing materials useful for the vaccination described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is one or more antibodies in a formulation of the invention as described above. The label on, or associated with, the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Therapeutic formulations are prepared for storage by mixing the vaccine having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The vaccine composition will be formulated, dosed, and administered in a fashion consistent with good medical practice. The “therapeutically effective amount” of the vaccine to be administered will be governed by clinical considerations, and is the minimum amount necessary to reduce virus titer in an infected individual.

One may adjust dosage based on the amount of peptide delivered. An immunologically effective dose is one that stimulates the immune system of the patient to establish a level immunological memory sufficient to provide long term protection against disease caused by infection. More precise dosages should be determined by assessing the immunogenicity of the vaccine produced so that an immunologically effective dose is delivered.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent, e.g. in the use of vaccine fragments, or in the use of vaccine conjugates. The dosage may also be varied for localized administration, or for systemic administration, e.g. i.m., i.p., i.v., and the like.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The invention now being fully described, it will be apparent to one of ordinary skill in the art that various changes and modifications can be made without departing from the spirit or scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all of the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

Prior research in our laboratory identified that Chlamydia species promote nonspecific B cell activation via synergistic activation of non-antigen-specific B cell receptors and TLR2-induced MyD88 signaling. We also showed such Chlamydia-activated B cells (CABs) can efficiently present cognate antigen to CD8⁺ T cells in vivo and that antigen-loaded CABs retain their capacity to stimulate T cell immunity after cryopreserved CABs are thawed. We also showed that loading CABs with cognate antigen and α-galactosylceramide (αGC), a type I NKT cell agonist, induced robust antigen-specific CD8⁺ T cell responses that reject established tumors and that one dose of intravenously administered CABs induce a large pool of hepatic CD8⁺ memory T cells. In mice, these hepatic CD8⁺ memory T cells also afforded complete protection against liver-stage malaria and added to our preclinical data demonstrating the ability of a CAB-based vaccine to activate potent antigen-specific CD8⁺ T cell responses in vivo. Maximal induction of in vivo CD8⁺ T cell responses was dependent on CAB utilization of the host's type 1 conventional dendritic cells (type 1 cDCs), and that this process was also independent of Clec9A, a classic cross-priming receptor.

The experiments described herein provide for improvements in technology to activate antigen-specific CD8⁺ T cell responses in vivo. This next generation vaccine platform reduces manufacturing costs and brings down logistical barrier associated with the production of a cellular vaccine.

CABs require host type 1 conventional dendritic cells (type 1 cDCs) for maximal induction of CD8⁺ T cell responses. In other words, they present antigen(s) to host dendritic cells and require host immunocompetency to optimize host immune responsiveness. Identifying which receptor CABs use for their interactions with dendritic cells provides identification of a target receptor novel receptor(s) capable of mediating cross-priming that can be exploited for vaccine development.

Cell microarray technology (Retrogenix) was used to identify specific human plasma membrane receptors that bind to human CABs. These screening studies utilized a library of expression vectors containing open reading frames encoding full-length human plasma membrane proteins.

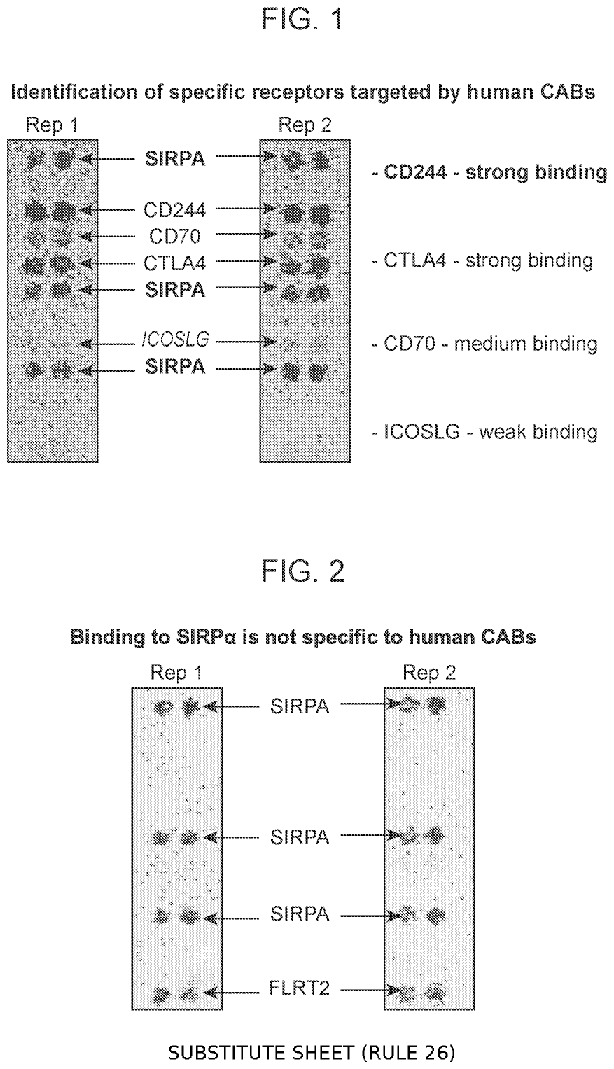

Four receptors with specific interactions to human CABs were identified (shown in FIG. 1). Two receptors were found to have strong binding to CABs: CD244 (other names: 2B4, SLAMF4) and CTLA4, one receptor was shown to have medium binding: CD70, and one receptor, ICOSLG, had weak binding. Binding of CABs to SIRPα was also identified in the screen, but it was shown to be non-specific as this interaction also happened with an irrelevant human cell line (FIG. 2). Importantly, this cell line did not bind to any of the receptors identified as specifically interacting with CABs.

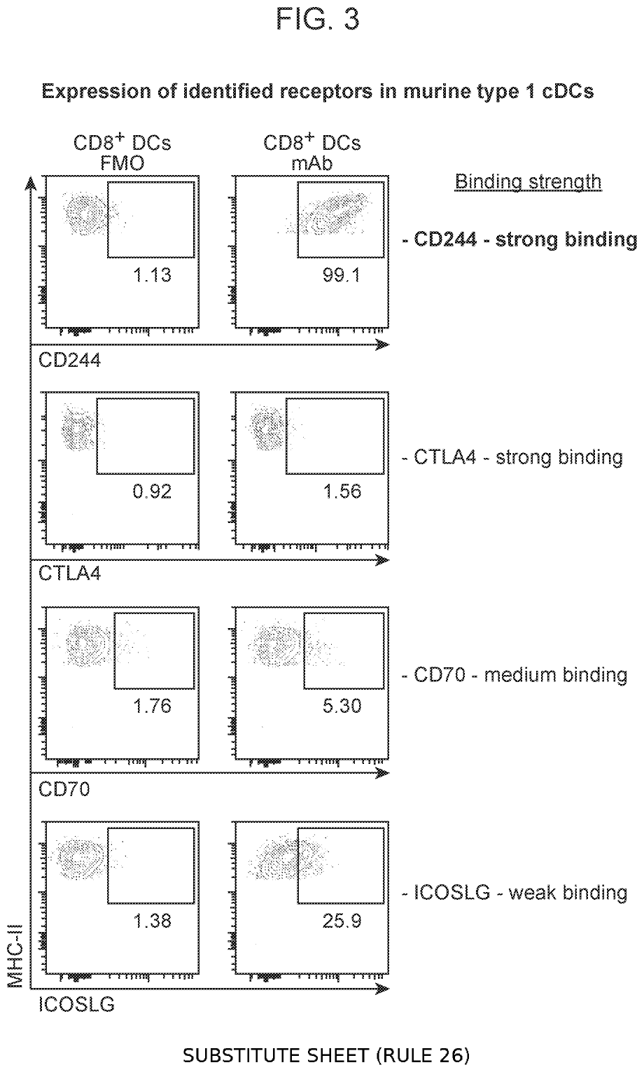

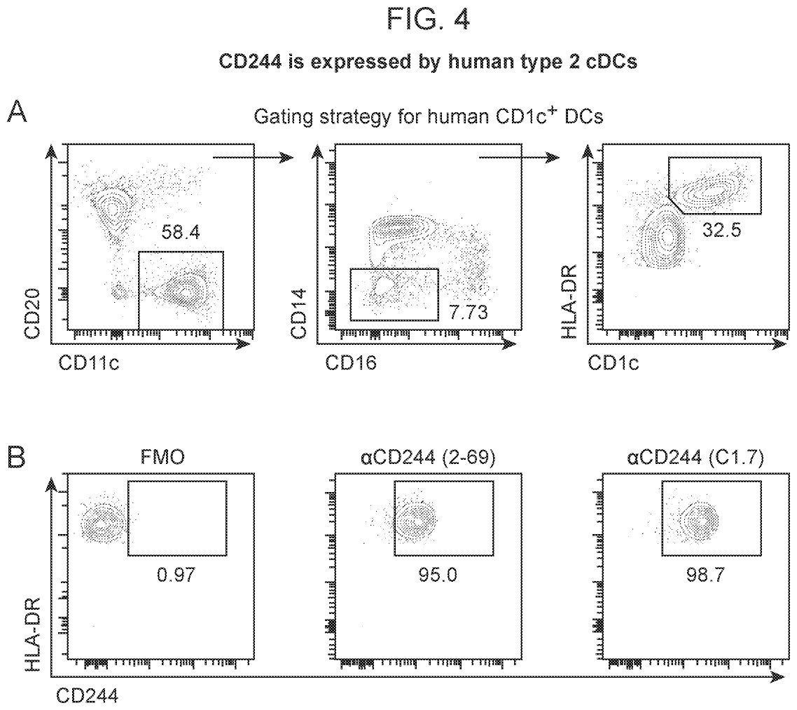

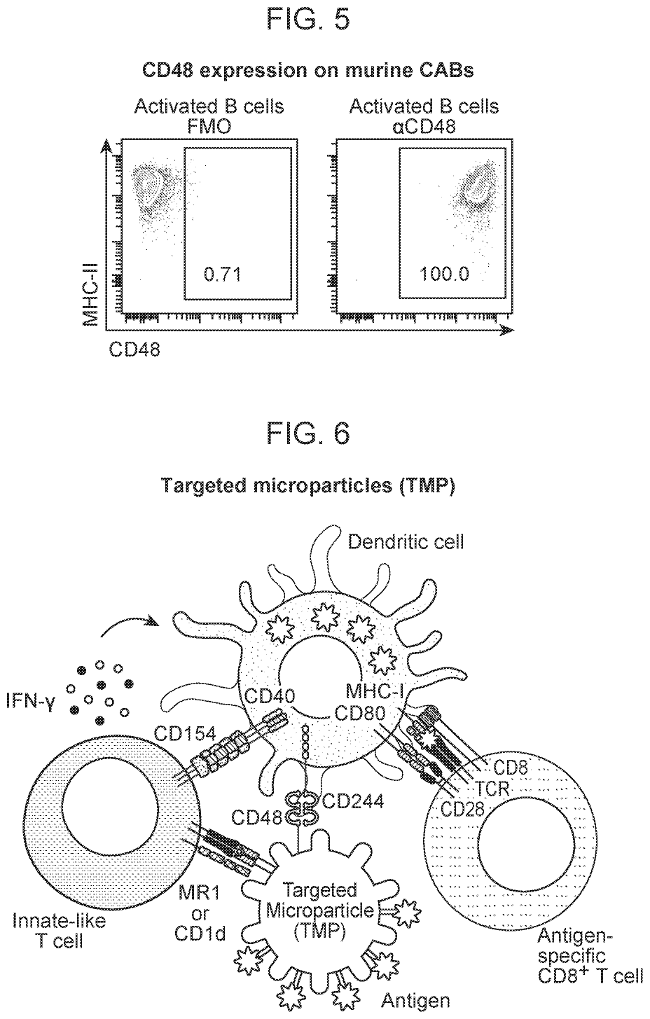

Both mice and human CABs were found to prime antigen-specific CD8⁺ T cell responses when administered intravenously to mice via a process dependent on the host's conventional dendritic cells. Initial evaluation of expression of the identified receptors in mouse DCs were performed with mouse cells. Splenic cDCs (type 1 and type 2) from C57BL/6 mice displayed high levels of CD244, intermediate levels of ICOSLG, and negligible levels of CTLA4 and CD70. Shown in FIG. 3 is expression of receptors in type 1 cDCs, but equivalent levels of expression were also seen in type 2 cDCs. Furthermore, human type 2 cDCs also express high levels of CD244 on their surface (FIG. 4). CD48 (also known as SLAMF2) is the only reported ligand for CD244. Since CABs were able to bind to CD244 in our screen, they were expected to express CD48, as shown in FIG. 5.

CABs induce robust antigen-specific CD8⁺ T cell responses in vivo by a combination of the following factors: binding to a receptor in DCs that allows them to deliver antigens for efficient cross-priming, ability to carry a sizable amount of antigen to these cells, and last, but critical for the promotion of the effector function of primed CD8⁺ T cells, their ability to activated innate-like T cells in vivo. In the mouse, activation of type I NKT cells was used to obtain this “adjuvant” effect since they are present at a higher frequency than other innate-like T cells in this animal model. However, recent data indicates that the role played by type I NKT cells in murine host defense has been evolutionarily adopted in nonhuman primates (NHPs) and humans by mucosal-associated invariant T (MAIT) cells. Analogous roles for hepatic NKT and MAIT cells, including the ability to activate dendritic cells, identify MAIT cells as an attractive new focus to activate innate-like T cells in humans and NHPs.

Targeted microparticles (TMPs) provide a platform that can have the same effectiveness as CABs at inducing antigen-specific T cell responses. TMPs are small particles, e.g. from about 0.1-3 μm in diameter, with a surface decorated with (i) a CD244 agonist, such as an antibody or ligand that bind to CD244, (ii) a sufficient dose of antigen to which a response is desired, and (iii) an activating moiety for innate-like T cells of interest. Such TMPs provide a cell-free and synthetic version of a CAB. A schematic depiction of our proposed approach is shown in FIG. 6. Further, antigens delivered in particulate form have increased immunogenicity compared to soluble antigens, as they can be taken up by dendritic cells by phagocytosis.

TMP synthesis. Commercially available polymer microspheres (Bang Laboratories) in combination with affinity binding systems (i.e., microspheres covered with streptavidin) offer efficient and straightforward ligand attachment (i.e., biotinylated proteins). We have tested microspheres of 3 μm and 0.2 μm in diameter, and have seen that 0.2 μm microspheres offer a bigger surface area in the same volume, increasing their binding capacity, and hence their activity in vivo. Further development can require a transition to biodegradable microparticles, for example poly(lactic-co-glycolic acid) (PLGA) microparticle formulations that allow easy incorporation of antigens inside the particles. PLGA-based TMPs require functionalization to allow CD244 agonist to be displayed on the surface.

Antigen selection varies with the desired response and varies depending on the clinical setting of interest. Initial experiments were performed with a whole protein, ovalbumin, but peptides or combinations of peptides as small as 8 amino acids in length can be used. Exemplary antigens include but are not limited to bacterial, viral, parasitic, allergens, autoantigens, and tumor-associated antigens. Particularly, the antigen can include protein antigens, peptides, whole inactivated organisms, and the like. Carbohydrates or lipids antigens are also applicable.

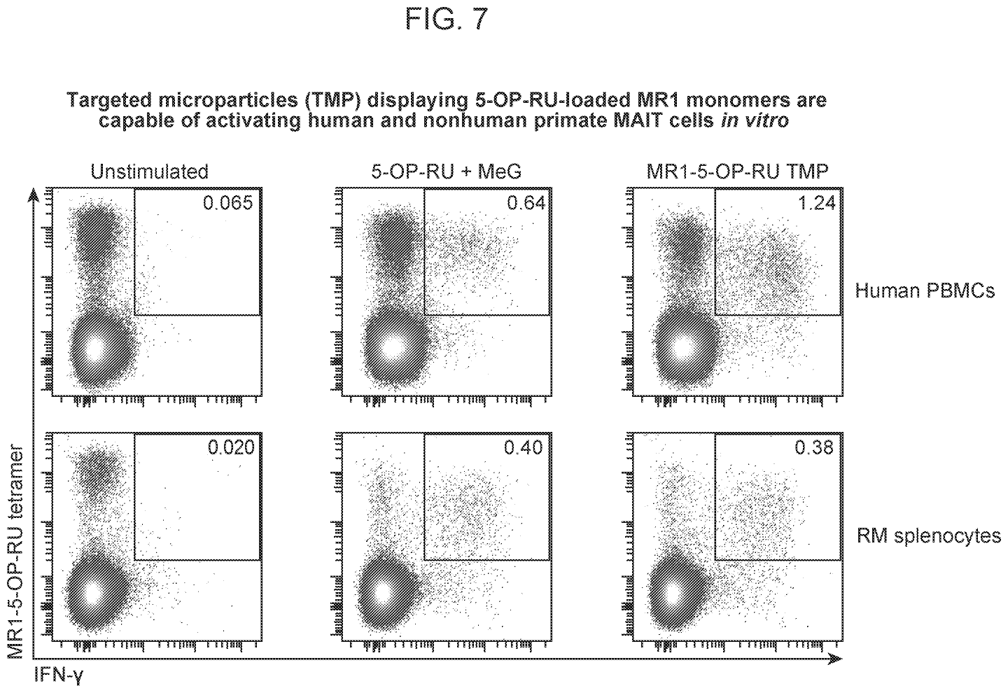

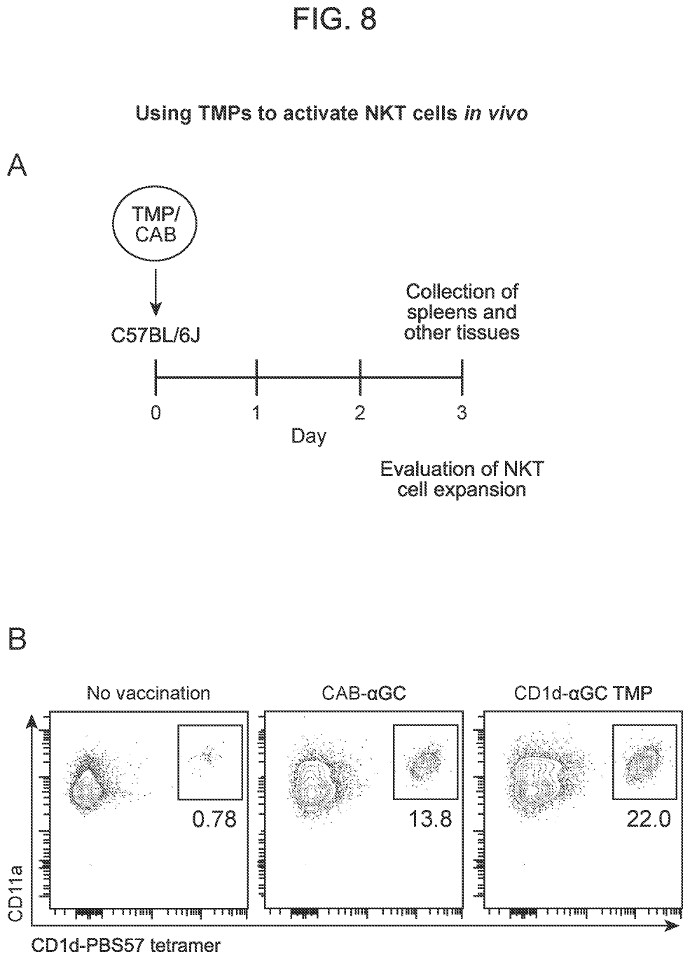

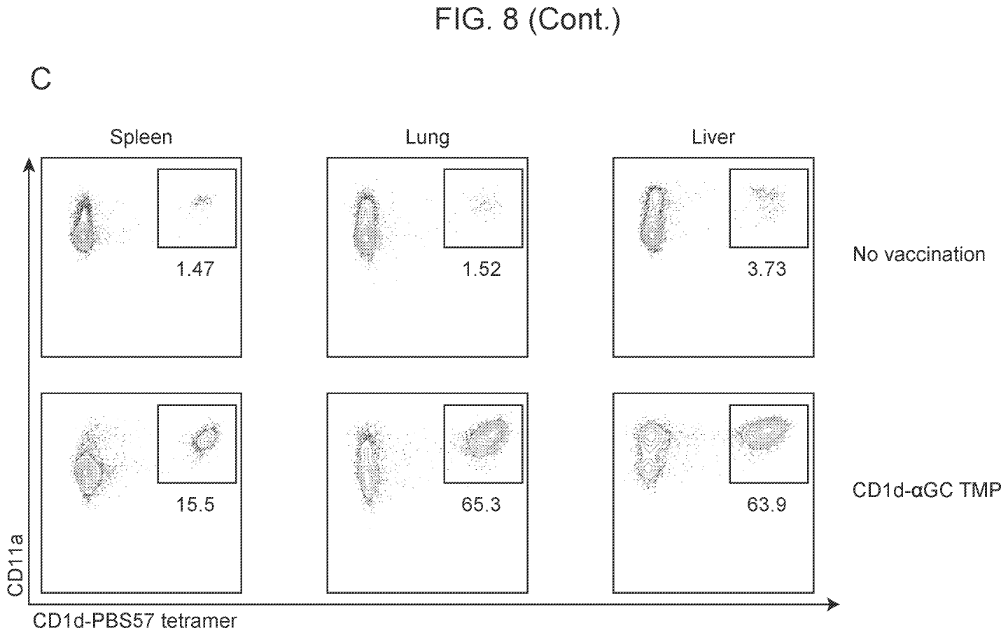

Activation of innate-like T cells and effector memory T cells can be achieved by presentation of their antigen in the context of the appropriate MHC molecule. Streptavidin-coated microspheres were linked to biotinylated monomers of CD1d (type I NKT cells) or MR1 (MAIT cells) to test for induction of activation of these innate-like T cells. As seen in FIG. 7, in vitro exposure of human and rhesus macaque MAIT cells to their cognate antigen (5-OP-RU), which is very unstable, or to microspheres loaded with 5-OP-RU-loaded MR-1 monomers was able to induce secretion of IFN-γ to a similar extent. We then performed an experiment in which mice were intravenously administrated CABs loaded with αGC (positive control), which would induce the expansion of NKT cells, CD1d-αGC-coated microspheres (test group), or were left untreated (negative control) (FIG. 8). As seen in FIG. 8B, a single dose of microspheres loaded with the appropriate innate-like T cell ligand was able to induce a massive expansion of NKT cells in vivo. Interestingly, this expansion of NKT cells was observed to a higher degree in liver and lungs, indicating that these two tissues are especially targeted by TMPs (FIG. 8C).

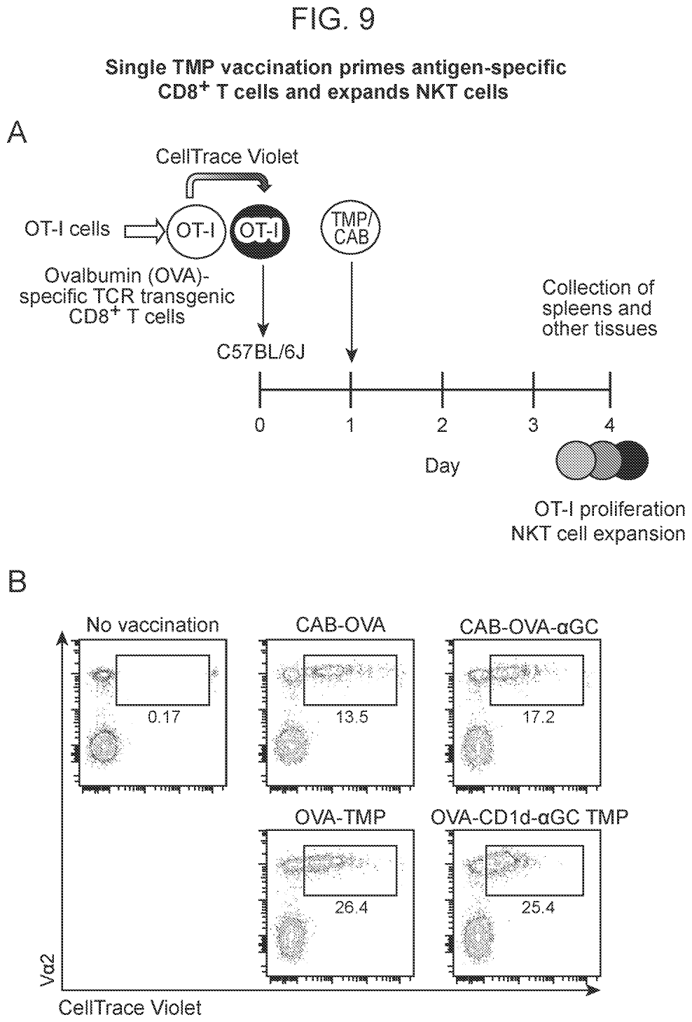

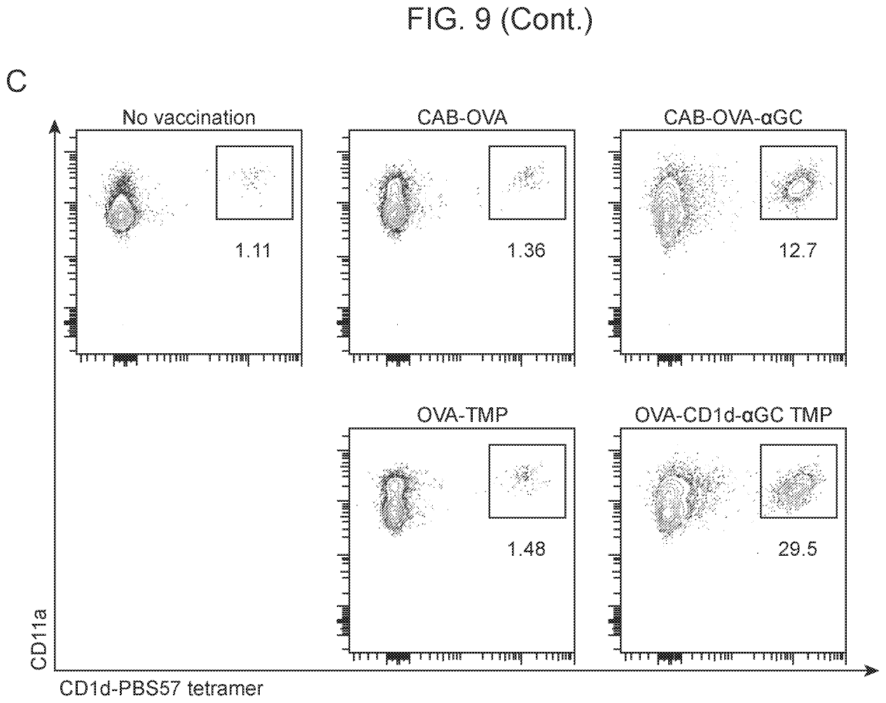

The combination of these components in a single TMP could provide priming of antigen-specific CD8⁺ T cells to a similar extent as that observed with CABs. An experimental system was used that takes advantage of the transfer of fluorescently (CTV)-labeled ovalbumin-specific CD8⁺ T cells (Vα2⁺ cells) from TCR-transgenic mice into regular C57BL/6 mice prior to the administration of antigen-loaded CABs that are also loaded or not with αGC, or TMPs covered with an anti-CD244 monoclonal antibody and loaded only with the antigen, or TMPs covered with an anti-CD244 monoclonal antibody and loaded with the antigen and with αGC-loaded CD1d monomers (FIG. 9). This experiment showed that CD244-targeted TMPs could prime antigen-specific CD8⁺ T cells responses, but that they could also provide the “adjuvant” activation of NKT cells needed for optimal effector function of primed CD8⁺ T cells (FIGS. 9B, 9C).

To activate innate-like T cells we used biotinylated CD1d monomers loaded with αGC or its analog PBS-57, or MR1 monomers loaded with 5-OP-RU.

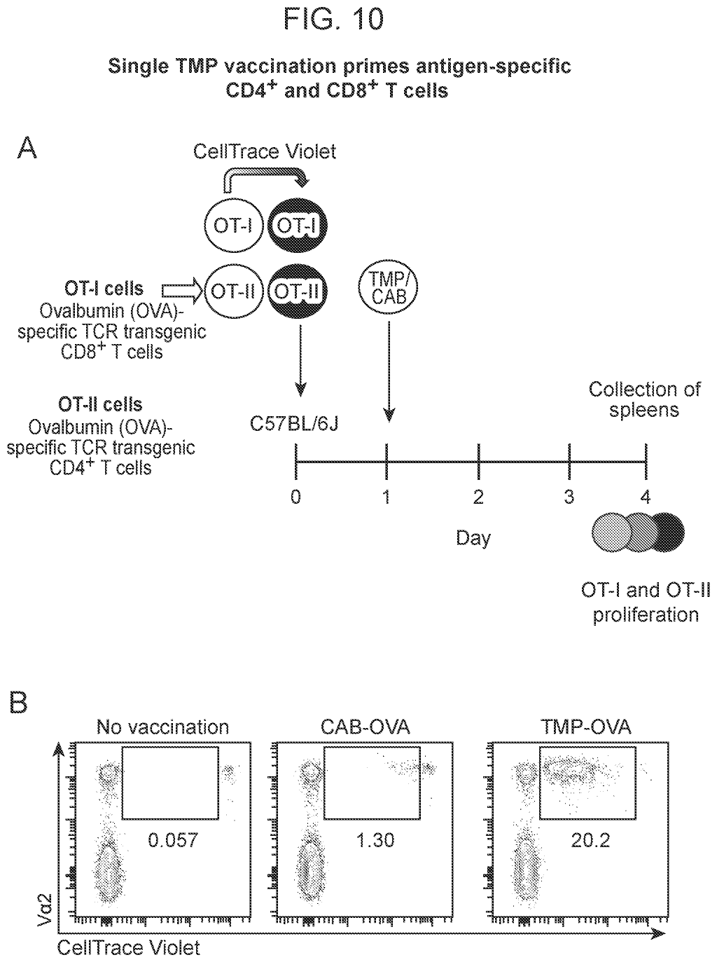

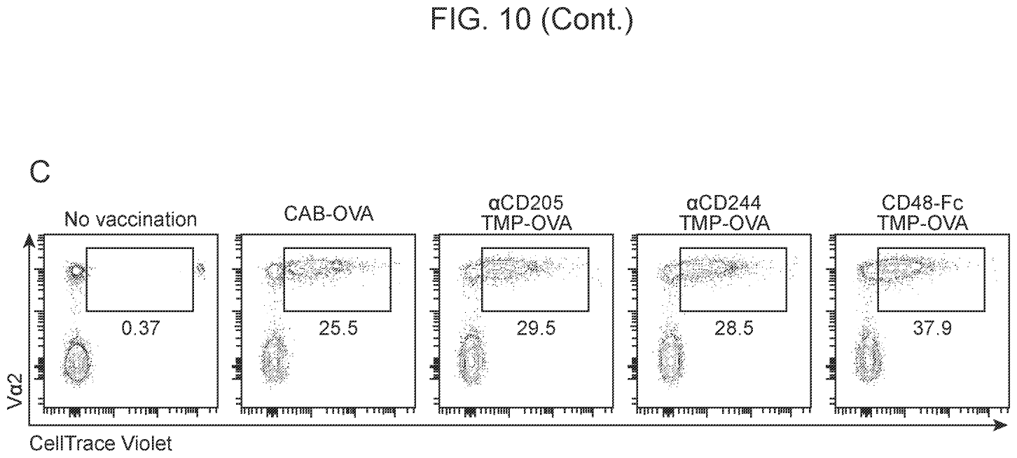

We have established that CABs can prime antigen-specific CD8⁺ T cells, but not CD4⁺ T cells in vivo. This may be a result of cell-associated antigen being exclusively shuttled through a cross-priming pathway in type 1 cDCs. Targeting CD244 should allow delivery of antigen(s) to both type 1 and type 2 cDCs, and microparticles should not have the same biological constraints that cell-associated antigens may have. To test this hypothesis, we used the same experimental model described above, but concomitantly transferred fluorescently-labeled ovalbumin-specific CD4⁺ T cells from TCR-transgenic mice (FIG. 10A). As expected, CABs were not able to prime CD4⁺ T cells in vivo; however, TMPs loaded with anti-CD244 mAb and antigen were able to induce robust proliferation of antigen-specific CD4⁺ T cells (FIG. 10B). The cell-free and synthetic vaccine platform represents an important advancement for a platform designed to induce optimal host antigen-specific immunity.

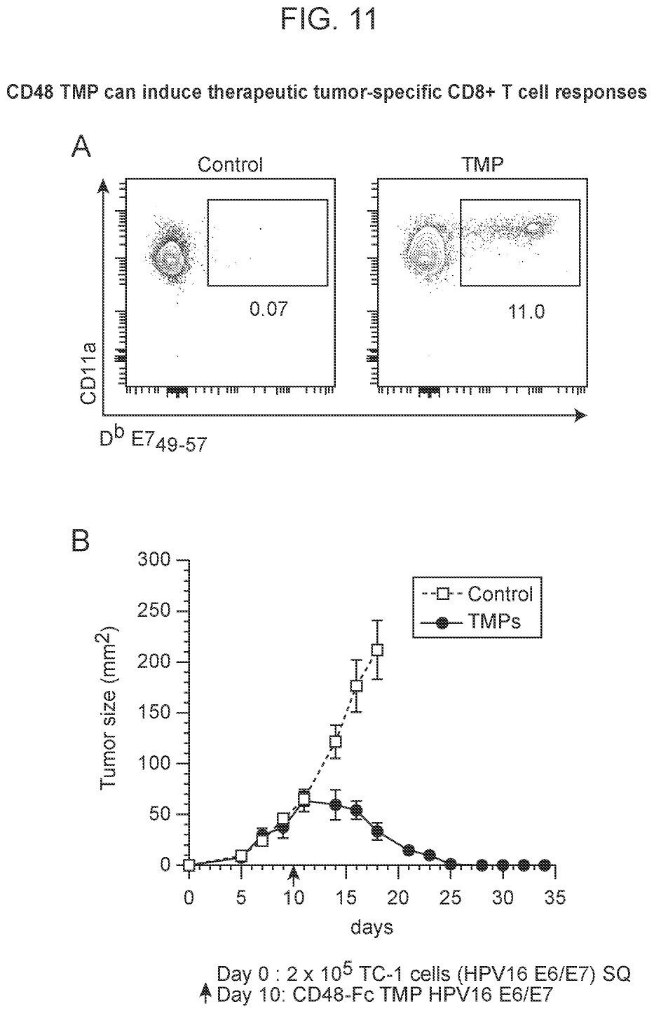

To compare TMPs loaded with anti-CD244 mAb vs. TMPs loaded with anti-CD205 (DEC205) mAb, a commonly used approach to target antigens to DCS in vivo, we performed experiments to determine priming of antigen-specific CD8⁺ T cells. In addition, we prepared TMPs loaded with recombinant mouse CD48 fused to human IgG1 to assess if they could provide advantages over an antibody-targeting approach. When we evaluated the proliferation of CD8⁺ T cells in response to the different vaccination approaches, we observed that anti-CD205 mAb-loaded TMPs and anti-CD244 mAb-loaded TMPs were able to similarly activate antigen-specific CD8⁺ T cells in vivo (FIG. 10C). Conversely, the use of natural ligand for targeting of TMPs to DCs demonstrated superior ability to promote antigen-specific CD8⁺ T cells in vivo (FIG. 11C).

We loaded TMPs with mouse CD48-Fc, CD1d monomers loaded with PBS-57 and the immunodominant epitope of HPV16 E7 (49-57) to demonstrate induction of therapeutic CD8⁺ T cell responses against a tumor-associated antigen. As shown in FIG. 11A, a single intravenous dose of these TMPs induced robust HPV16 E7-specific CD8⁺ T cell responses in mice without tumors. Furthermore, TMPs loaded with mouse CD48-Fc, CD1d monomers loaded with PBS-57 and the immunodominant epitopes of HPV16 E6 (48-57) and E7 (49-57) were able to induce the complete rejection of established tumors in a mouse model of HPV-associated tumors (FIG. 11B).

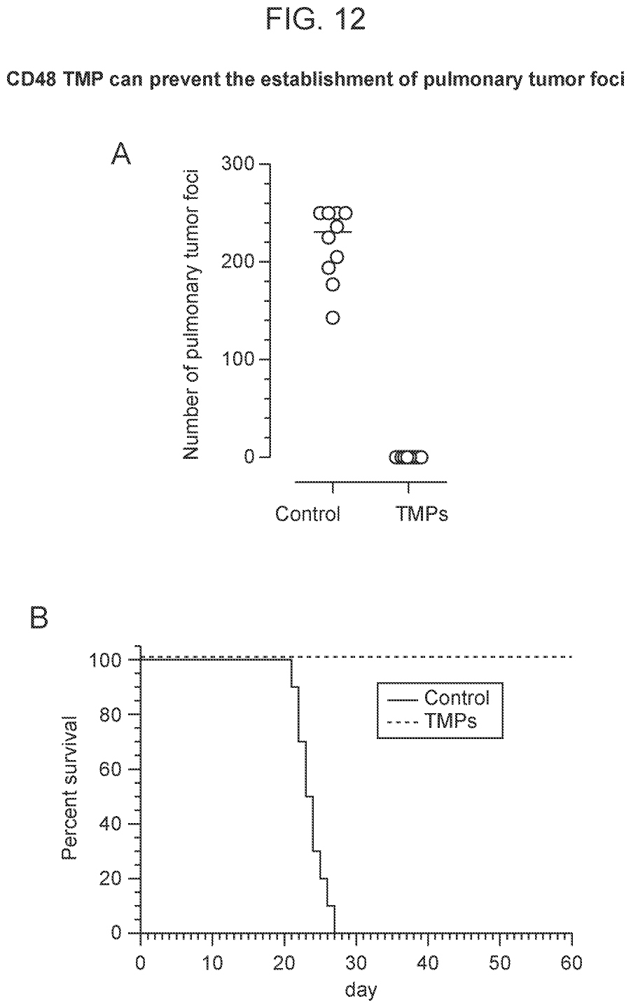

Vaccination of mice with a single dose of TMPs with mouse CD48-Fc, CD1d monomers loaded with PBS-57 and 2 melanoma-associated epitopes (TRP2 and gp100) was able to prevent establishment of pulmonary tumor foci after the injection of B16-F10 melanoma cells (FIG. 12).

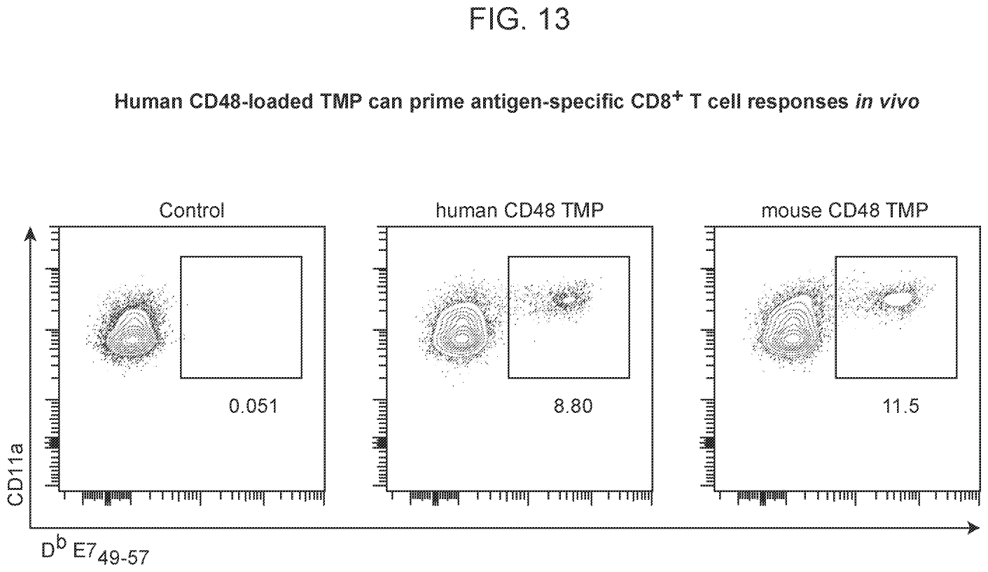

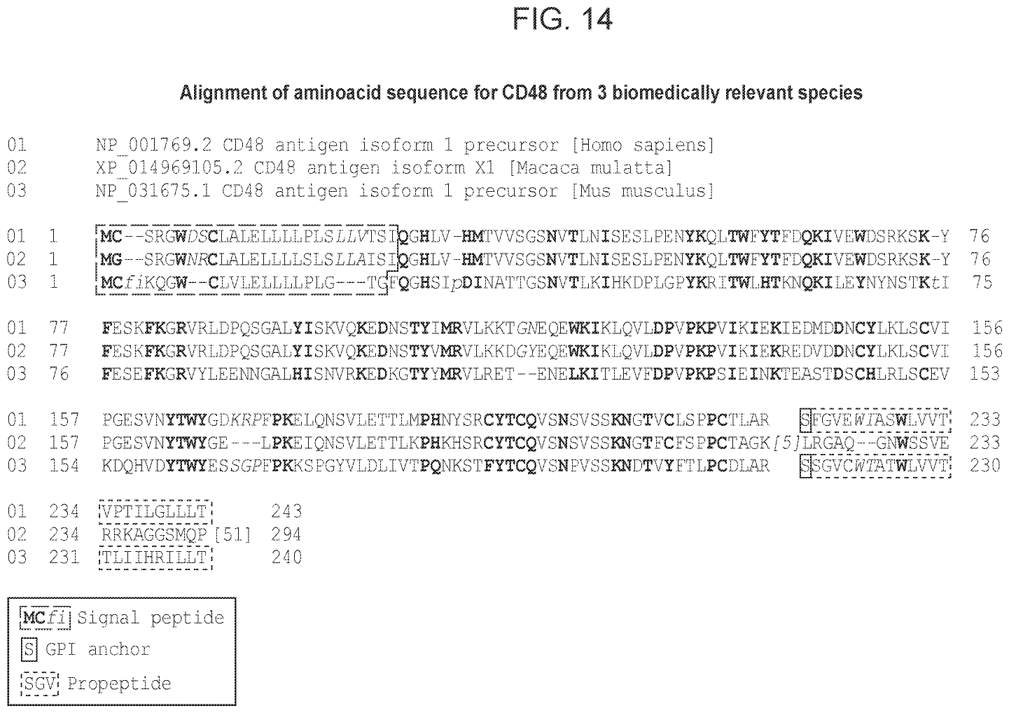

As mentioned before, both mouse and human CABs can prime antigen-specific CD8⁺ T cell responses in mice. We took advantage of this experimental system to test if TMPs loaded with human CD48 fused to human IgG1 or mouse CD48 with a Hist tag, and also concomitantly loaded with CD1d monomers loaded with PBS-57 and the immunodominant epitope of HPV16 E7 (49-57), can induce antigen-specific CD8⁺ T cells in vivo. As shown in FIG. 13, human CD48-decorated were capable of inducing antigen-specific CD8⁺ T cells after intravenous administration into non-tumor bearing mice.

The TMPs are being tested on a mouse model of liver-stage malaria. Preclinical studies will be performed to test their immunogenicity in nonhuman primates. Developing a synthetic vaccine capable of inducing vigorous CD8⁺ T cell responses after a single administration has the potential to revolutionize malaria vaccine development, as the first-ever human CD8⁺ T cell vaccine.

In conclusion, we disclose a novel method for targeting antigen-presenting cells through a specific receptor, CD244 (2B4, SLAMF4), using antibodies against the receptor or recombinant versions of the natural ligand of the receptor, CD48 (SLAMF2). The targeting of antigens to CD244 using microparticles results in efficient antigen presentation in the context of MHC-I and MHC-II, leading to the robust priming of CD8⁺ and CD4⁺ T cells, respectively. The inclusion of an activator of innate-like T cells (e.g., CD1d monomers loaded with αGC or MR1) leads to the increased expansion and effector function of responding T cells, and also to the expansion of the stimulated innate-like T cells after a single intravenous administration of TMPs.

The ability of TMPs to induce antigen-specific CD4⁺ and CD8⁺ T cell responses in vivo opens the door to applications beyond clinical conditions in which CD8⁺ T cell responses (e.g., tumors) are need to combat disease to clinical conditions that also benefit from the generation of robust antigen-specific CD4⁺ T cell responses (e.g., tuberculosis).

Sequences

SEQ ID NO: 1 Human CD48 protein
MCSRGWDSCLALELLLLPLSLLVTSIQGHLVHMTVVSGSNVTLNISESLPENYKQLTWFYTFDQKIVEWD
SRKSKYFESKFKGRVRLDPQSGALYISKVQKEDNSTYIMRVLKKTGNEQEWKIKLQVLDPVPKPVIKIEKI
EDMDDNCYLKLSCVIPGESVNYTWYGDKRPFPKELQNSVLETTLMPHNYSRCYTCQVSNSVSSKNGTV
CLSPPCTLARSFGVEWIASWLVVTVPTILGLLLT
SEQ ID NO: 2 macaque CD48 sequence
MGSRGWNRCLALELLLLSLSLLAISIQGHLVHMTVVSGSNVTLNISESLPENYKQLTWFYTFDQKIVEWDS
RKSKYFESKFKGRVRLDPQSGALYISKVQKEDNSTYVMRVLKKDGYEQEWKIKLQVLDPVPKPVIKIEKR
EDVDDNCYLKLSCVIPGESVNYTWYGELPKEIQNSVLETTLKPHKHSRCYTCQVSNSVSSKNGTFCFSP
PCTAGKLRGAQGNWSSVERRKAGGSMQP
SEQ ID NO: 3 mouse CD38 sequence
MCfiKQGWCLVLELLLLPLGTGFQGHSlpDINATTGSNVTLKIHKDPLGPYKRITWLHTKNQKILEYNYNSTK
tIFESEFKGRVYLEENNGALHISNVRKEDKGTYYMRVLRETENELKITLEVFDPVPKPSIEINKTEASTDSC
HLRLSCEVKDQHVDYTWYESSGPFPKKSPGYVLDLIVTPQNKSTFYTCQVSNPVSSKNDTVYFTLPCDL
ARSSGVCWTATWLVVTTLIIHRILLT
SEQ ID NO: 4 human CD48-Fc chimera
MCSRGWDSCLALELLLLPLSLLVTSIQGHLVHMTVVSGSNVTLNISESLPENYKQLTWFYTFDQKIVEWD
SRKSKYFESKFKGRVRLDPQSGALYISKVQKEDNSTYIMRVLKKTGNEQEWKIKLQVLDPVPKPVIKIEKI
EDMDDNCYLKLSCVIPGESVNYTWYGDKRPFPKELQNSVLETTLMPHNYSRCYTCQVSNSVSSKNGTV
CLSPPCTLARSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGK
Human CD48 residues 1-220; Human IgG1 residues 221-550
SEQ ID NO: 5 human CD48-Fc chimera
QGHLVHMTVVSGSNVTLNISESLPENYKQLTWFYTFDQKIVEWDSRKSKYFESKFKGRVRLDPQSGALY
ISKVQKEDNSTYIMRVLKKTGNEQEWKIKLQVLDPVPKPVIKIEKIEDMDDNCYLKLSCVIPGESVNYTWY
GDKRPFPKELQNSVLETTLMPHNYSRCYTCQVSNSVSSKNGTVCLSPPCTLARSIEGRMDASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
Human CD48 residues 1-194; Linker residues 195-200; Human IgG1 residues 201-530
SEQ ID NO: 6 human CD48 his-tag
MCSRGWDSCLALELLLLPLSLLVTSIQGHLVHMTVVSGSNVTLNISESLPENYKQLTWFYTFDQKIVEWD
SRKSKYFESKFKGRVRLDPQSGALYISKVQKEDNSTYIMRVLKKTGNEQEWKIKLQVLDPVPKPVIKIEKI
EDMDDNCYLKLSCVIPGESVNYTWYGDKRPFPKELQNSVLETTLMPHNYSRCYTCQVSNSVSSKNGTV
CLSPPCTLARSHHHHHH
Human CD48 residues 1-220; His-tag residues 221-227
SEQ ID NO: 7 human CD48 his-tag protein
QGHLVHMTVVSGSNVTLNISESLPENYKQLTWFYTFDQKIVEWDSRKSKYFESKFKGRVRLDPQSGALY
ISKVQKEDNSTYIMRVLKKTGNEQEWKIKLQVLDPVPKPVIKIEKIEDMDDNCYLKLSCVIPGESVNYTWY
GDKRPFPKELQNSVLETTLMPHNYSRCYTCQVSNSVSSKNGTVCLSPPCTLARSHHHHHHHHHH
Human CD48 residues 1-194; His-tag residues 195-204
SEQ ID NO: 8 human CD48 protein with flexible linker
QGHLVHMTVVSGSNVTLNISESLPENYKQLTWFYTFDQKIVEWDSRKSKYFESKFKGRVRLDPQSGALY
ISKVQKEDNSTYIMRVLKKTGNEQEWKIKLQVLDPVPKPVIKIEKIEDMDDNCYLKLSCVIPGESVNYTWY
GDKRPFPKELQNSVLETTLMPHNYSRCYTCQVSNSVSSKNGTVCLSPPCTLARSGSAGSAAGSGEFHH
HHHH
Human CD48 residues 1-194; Flexible linker residues 195-206; His-tag residues 207-212
SEQ ID NO: 9 mouse CD48-Fc protein
MCFIKQGWCLVLELLLLPLGTGFQGHSIPDINATTGSNVTLKIHKDPLGPYKRITWLHTKNQKILEYNYNST
KTIFESEFKGRVYLEENNGALHISNVRKEDKGTYYMRVLRETENELKITLEVFDPVPKPSIEINKTEASTDS
CHLRLSCEVKDQHVDYTWYESSGPFPKKSPGYVLDLIVTPQNKSTFYTCQVSNPVSSKNDTVYFTLPCD
LARASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
Mouse CD48 residues 1-216; Human IgG1 residues 217-546
SEQ ID NO: 10 Mouse CD48-Fc Protein
FQGHSIPDINATTGSNVTLKIHKDPLGPYKRITWLHTKNQKILEYNYNSTKTIFESEFKGRVYLEENNGALH
ISNVRKEDKGTYYMRVLRETENELKITLEVFDPVPKPSIEINKTEASTDSCHLRLSCEVKDQHVDYTWYES
SGPFPKKSPGYVLDLIVTPQNKSTFYTCQVSNPVSSKNDTVYFTLPCDLARIEGRMDASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
Mouse CD48 residues 1-194, Linker residues 195-200; Human IgG1 residues 201-530
SEQ ID NO: 11 Mouse CD48 His-tag Protein
MCFIKQGWCLVLELLLLPLGTGFQGHSIPDINATTGSNVTLKIHKDPLGPYKRITWLHTKNQKILEYNYNST
KTIFESEFKGRVYLEENNGALHISNVRKEDKGTYYMRVLRETENELKITLEVFDPVPKPSIEINKTEASTDS
CHLRLSCEVKDQHVDYTWYESSGPFPKKSPGYVLDLIVTPQNKSTFYTCQVSNPVSSKNDTVYFTLPCD
LARHHHHHH
Mouse CD48 residues 1-216; His-tag residues 217-222
SEQ ID NO: 12 Mouse CD48 His-tag Protein
FQGHSIPDINATTGSNVTLKIHKDPLGPYKRITWLHTKNQKILEYNYNSTKTIFESEFKGRVYLEENNGALH
ISNVRKEDKGTYYMRVLRETENELKITLEVFDPVPKPSIEINKTEASTDSCHLRLSCEVKDQHVDYTWYES
SGPFPKKSPGYVLDLIVTPQNKSTFYTCQVSNPVSSKNDTVYFTLPCDLARHHHHHH
Mouse CD48 residues 1-194; His-tag residues 195-200
SEQ ID NO: 13 Mouse CD48 with linker
FQGHSIPDINATTGSNVTLKIHKDPLGPYKRITWLHTKNQKILEYNYNSTKTIFESEFKGRVYLEENNGALH
ISNVRKEDKGTYYMRVLRETENELKITLEVFDPVPKPSIEINKTEASTDSCHLRLSCEVKDQHVDYTWYES
SGPFPKKSPGYVLDLIVTPQNKSTFYTCQVSNPVSSKNDTVYFTLPCDLARGSAGSAAGSGEFHHHHH
Mouse CD48 residues 1-194; Flexible linker residues 195-206; His-tag residues 207-212

1. A vaccine composition comprising:
(i) an agent that specifically binds to CD244; (ii) an effective dose of an antigen; and (iii) an adjuvant.

2. The vaccine composition of claim 1, wherein the adjuvant is an activator of innate-like T cells.

3. The vaccine composition of claim 1, where the composition is a particle comprising each of components (i), (ii), and (iii).

4. The vaccine composition of claim 1, wherein administration of the vaccine composition to a mammalian subject enhances T cell responsiveness to the (ii) antigen.

5. The vaccine composition of claim 1, wherein the enhanced T cell responses are one or both of antigen-specific CD4⁺ T cell responses and antigen-specific CD8⁺ T cell responses.

6. The vaccine composition of claim 1, wherein the agent that specifically binds to CD244 is an antibody.

7. The vaccine composition of claim 6, wherein the antibody is an intact antibody.

8. The vaccine composition of claim 6 wherein the antibody is a fragment comprising a variable region domain.

9. The vaccine composition of claim 1, wherein the agent that specifically binds to CD244 is CD48 or a binding fragment derived therefrom.

10. The vaccine composition of claim 9, wherein CD48 is human CD48.

11. The vaccine composition of claim 1, wherein the antigen is a polypeptide antigen.

12. The vaccine composition of claim 11, wherein the antigen is a tumor antigen.

13. The vaccine composition of claim 11, wherein the antigen is a pathogen antigen.

14. The vaccine composition of claim 1, wherein the activator of innate-like T cells is an MHC-related protein and antigen recognized by the targeted population of innate-like T cells.

15. The vaccine composition of claim 14, wherein the innate-like T cells are mucosal-associated invariant T (MAIT) cells; and the MHC-related protein is MR1 complexed with a microbial derived metabolite or analog thereof.

16. The vaccine composition of claim 14, wherein the innate-like T cells are invariant natural killer T (iNKT) cells, and the MHC-related protein is CD1d complexed with α-galactosylceramide.

17. The vaccine composition of claim 1, comprising a biodegradable microparticle comprising each of components (i), (ii), and (iii).

18. The vaccine composition of claim 17, wherein each of (i), (ii), and (iii) is encapsulated within the biodegradable microparticle.

19. The vaccine composition of claim 17, wherein component (i) is displayed on the surface of the microparticle.

20. The vaccine composition of claim 1 wherein the biodegradable microparticle is from about 0.1 μm in diameter to about 5 μm in diameter.

21. The vaccine composition of claim 1, wherein the microparticle is comprised of poly (lactic acid) (PLA), poly(glycolic acid) (PGA), or a combination thereof (PLGA).

22. A method of stimulating a T cell response to an antigen of interest, the method comprising:
administering to an individual mammal an effective dose or series of doses of a vaccine composition according to claim 1 in a dose and frequency sufficient to induce a protective immune response.


SEQUENCE LISTING (13 sequences)

SEQ ID NO: 1 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 2 - protein, 238 aa, Macaca mulatta
MGSRGWNRCLALELLLLSLSLLAISIQGHLVHMTVVSGSNVTLNISESLPENYKQLTWFYTFDQKIVEWDSRKSKYFESKFKGRVRLDPQSGALYISKVQKEDNSTYVMRVLKKDGYEQEWKIKLQVLDPVPKPVIKIEKREDVDDNCYLKLSCVIPGESVNYTWYGELPKEIQNSVLETTLKPHKHSRCYTCQVSNSVSSKNGTFCFSPPCTAGKLRGAQGNWSSVERRKAGGSMQP

SEQ ID NO: 3 - protein, 240 aa, Mus musculus
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSSGVCWTATWLVVTTLIIHRILLT

SEQ ID NO: 4 - protein, 550 aa, Artificial sequence
  human CD48-Fc chimera
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 530 aa, Artificial sequence
  human CD48-Fc chimera
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSIEGRMDASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 226 aa, Artificial sequence
  human CD48 his-tag
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSHHHHHH

SEQ ID NO: 7 - protein, 204 aa, Artificial sequence
  human CD48 his-tag protein
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSHHHHHHHHHH

SEQ ID NO: 8 - protein, 212 aa, Artificial sequence
  human CD48 protein with flexible linker
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSGSAGSAAGSGEFHHHHHH

SEQ ID NO: 9 - protein, 546 aa, Artificial sequence
  mouse CD48-Fc protein
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 530 aa, Artificial sequence
  Mouse CD48-Fc Protein
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARIEGRMDASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 222 aa, Artificial sequence
  Mouse CD48 His-tag Protein
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARHHHHHH

SEQ ID NO: 12 - protein, 200 aa, Artificial sequence
  Mouse CD48 His-tag Protein
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARHHHHHH

SEQ ID NO: 13 - protein, 212 aa, Artificial sequence
  Mouse CD48 with linker
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARGSAGSAAGSGEFHHHHHH